(19) United States
(12) Patent Application Publication
(10) Pub. No.: US 2013/0090640 A1
(43) Pub. Date: Apr. 11, 2013

(54) METHODS AND SYSTEMS FOR DETECTION AND THERMAL TREATMENT OF LOWER URINARY TRACT CONDITIONS

(71) Applicants: Boston Scientific Scimed, Inc., Maple Grove, MN (US); University of Surrey, Surrey (GB)

(72) Inventors: Sandra Nagale, Lowell, MA (US); Ruth Cheng, Natick, MA (US); John Sherry, Needham, MA (US); David Borzelleca, Wexford, PA (US); Theresa Hague, Surrey (GB); John Young, Hampshire (GB); Christopher H. Fry, Surrey (GB)

(73) Assignees: University of Surrey, Surrey (GB); Boston Scientific Scimed, Inc., Maple Grove, MN (US)

(21) Appl. No.: 13/646,362

(22) Filed: Oct. 5, 2012

Related U.S. Application Data
(60) Provisional application No. 61/544,878, filed on Oct. 7, 2011.
(60) Provisional application No. 61/584,510, filed on Jan. 9, 2012.
(60) Provisional application No. 61/618,429, filed on Mar. 30, 2012.

(51) Int. Cl. A61B 18/04

(52) U.S. Cl. 606/27

(57) ABSTRACT

The invention is directed to a method for treating a urinary system. The method includes positioning one or more treatment units with respect to an organ wall. The one or more treatment units may be configured to deliver thermal energy. The method further includes delivering energy through the one or more treatment units to heat tissue of the organ wall.

### CROSS-REFERENCE TO RELATED APPLICATIONS

This application claims the benefit of priority from U.S. Provisional Application No. 61/544,878, filed Oct. 7, 2011, U.S. Provisional Application No. 61/584,510, filed Jan. 9, 2012, and U.S. Provisional Application No. 61/618,429, filed Mar. 30, 2012, the entirety of all of which are incorporated herein by reference.

### FIELD OF THE INVENTION

Embodiments of the invention relate generally to systems and methods for detecting and/or treating lower urinary tract conditions. More particularly, embodiments of the invention relate to systems and methods for detecting and/or treating bladder overactivity.

### BACKGROUND OF THE INVENTION

Overactive bladder is characterized by involuntary contractions of the detrusor muscle during bladder filling, which result in a sudden urge to urinate. The urge may be difficult to suppress, and can lead to involuntary loss of urine. Clinical manifestations of detrusor instability include urinary frequency, urinary urgency, and urinary urge incontinence.

Though little is known about the mechanisms underlying detrusor instability, recent studies suggest that the abnormal activity of the detrusor muscle may be a consequence of changes in the morphology and physiological or biochemical function of nerve, muscle, and connective tissues. These changes likely originate from defects on the cellular level or from changes in the nervous system. See M. J. Drake et al., Model of peripheral autonomous modules and a myovesical plexus in normal and overactive bladder function, 350 The Lancet 401, 401-403 (2001). Morphological studies show that changes to the nerve, muscle, and connective tissues are not uniform in idiopathic and neuropathic bladders. Instead, discrete areas of connective tissue infiltration, muscle hypertrophy, and altered innervations have been observed. See R. G. Charlton et al., Focal changes in nerve, muscle and connective tissue in normal and unstable human bladder, 84 BJU Int. 953, 953-960 (1999). These localized changes in the morphology of bladder tissue may contribute to abnormal function of the detrusor muscle on a macroscopic scale.

Moreover, studies suggest that the abnormal activity of the detrusor muscle may originate from one or more distinct anatomical areas of the bladder. For example, the abnormal activity of the detrusor muscle may originate in either the bladder dome or the internal sphincter, resulting in the dyssynchronous function of the entire bladder. In some instances, abnormal activity of the detrusor muscle may originate in the trigone. Evidence of cellular communication between the trigone and the detrusor muscle suggests that spontaneous activity of the trigone may be a precursor to bladder overactivity. See A. Roosen et al., Characteristics of Spontaneous Activity in the Bladder Trigone, 56 European Urology 346, 346-354 (2009).

Current methods to treat bladder overactivity include systemic drugs, nerve stimulation, and electrical stimulation. These known methods target the function of the entire bladder and do not address local changes and activity at specific anatomical areas of the bladder. Therefore, a need exists for methods and systems capable of both identifying and/or delivering therapy to specific anatomical areas of the bladder.

### SUMMARY OF THE INVENTION

Embodiments of the present invention are directed to systems and methods for detecting and/or treating lower urinary tract conditions that obviate one or more of the limitations and disadvantages of known methods.

One embodiment of the invention is directed to a medical device. The medical device may include an elongate member having a proximal end and a distal end and an end effector assembly that extends distally from the distal end of the elongate member. The end effector assembly may include a plurality of end effector units. Each end effector unit may have a sensing element for detecting a location of abnormal organ function and a treatment element for treating the location of abnormal organ function.

In various embodiments, the medical device may include one or more of the following additional features: wherein the end effector assembly includes a plurality of legs extending from a proximal end of the end effector assembly to the distal end of the end effector assembly, the plurality of legs forming a three-dimensional sphere in an expanded state; wherein each leg includes a plurality of lumens, and wherein each lumen is in communication with a corresponding lumen of the elongate member; wherein each lumen of each leg of the end effector assembly and corresponding lumen of the elongate member receives a bundle that terminates at one of the plurality of end effectors; wherein the bundle includes a wire, a fluid conduit, a lead, a catheter, and an optical fiber; wherein the plurality of end effector units are substantially uniformly disposed on the end effector assembly; wherein each end effector unit further includes a second treatment element for treating the location of abnormal organ function; and wherein each end effector unit further includes a fluid conduit for delivering a fluid to the location of abnormal organ function.

Another embodiment of the invention is directed to a device for treating a lower urinary tract condition. The device may include an elongate member having a proximal end, a distal end, and one or more lumens. The device may also include an end effector assembly extending distally from the distal end of the elongate member. The end effector assembly may define one or more exit apertures. Each exit aperture may be in communication with a corresponding lumen of the elongate member. The device may also include one or more bundles. Each bundle may extend through one of the one or more lumens of the elongate member and terminate at an end effector unit disposed in one of the one or more exit apertures.

In various embodiments, the system may include one or more of the following additional features: wherein the end effector unit is configured to contact tissue; wherein the end effector unit includes one or more apertures and a fluid port; wherein each bundle includes a wire that terminates at a sensing device provided in one of the one or more apertures of the end effector unit; wherein each bundle includes an energy transmission medium that terminates at a treatment element provided in another of the one or more apertures of the end effector unit; and wherein the treatment element is adjacent a distal facing surface of the end effector unit; and wherein each bundle includes a fluid conduit that terminates at the fluid port.

Yet another embodiment of the invention is directed to a method for treating a urinary system. The method may include inserting a medical device within a vesicle of the urinary system tract. The medical device may include an elongate member having a proximal end and a distal end and an end effector assembly extending distally from the distal end of the elongate member. The end effector assembly may include a plurality of end effector units each having a sensing element for detecting a location of abnormal function and a treatment element for treating the location of abnormal function. The method may further include positioning the medical device adjacent tissue of the vesicle of the urinary system and treating the location with the treatment element.

In various embodiments, the method may include one or more of the following additional features: further including detecting a location of abnormal function; contacting tissue with the plurality of end effector units; wherein the step of treating the location includes delivering energy through all of the end effector units; and further comprising delivering therapeutic fluids to tissue at the location treated with thermal energy; wherein the treatment element is configured to treat only a single layer of tissue at the location of abnormal organ function; wherein the treatment element is configured to treat only a mucosal layer of tissue; further including selectively treating only a single layer of tissue at the location of abnormal function; wherein the layer of tissue is a mucosal layer of tissue; further including delivering energy from the treatment element, and controlling a depth of thermal treatment to target only a single layer of tissue at the location of abnormal function; further including delivering energy from the treatment element, and controlling a temperature of thermal treatment to target a layer of tissue at the location of abnormal function; further including delivering energy from the treatment element at a predetermined temperature and targeting a desired depth of tissue.

Yet another embodiment of the invention is directed to a method for treating a urinary system. The method may include inserting a medical device within an organ of the urinary system. The medical device may include a support structure configured to conform to a portion of the organ. The plurality of treatment elements may be disposed on the support structure for treating a location of abnormal organ function. The method may further include positioning the medical device on the organ so that the plurality of treatment elements contact the organ; and treating the location with the treatment element.

In various embodiments, the method may include one or more of the following additional features: further including delivery energy through all of the treatment elements; delivering energy through the treatment elements to treat only a single layer of tissue at the location of abnormal organ function; wherein the single layer is a muscular layer of tissue; wherein the single layer is a mucosal layer of tissue; further including delivery energy through the treatment elements to increase a local temperature of only a single layer of tissue at the location of abnormal organ function; wherein the single layer is a muscular layer of tissue; wherein the single layer of tissue is a mucosal layer of tissue; and further including targeting a desired depth of tissue and delivering energy from the treatment element to achieve a predetermined temperature at the desired depth.

Yet another embodiment of the invention is directed to a method for treating a urinary system. The method may include positioning one or more treatment units with respect to an organ wall, the one or more treatment units being configured to deliver thermal treatment. The method may further include delivering energy through the one or more treatment units to heat tissue of the organ wall.

In various embodiments, the method may include one or more of the following additional features: further including heating only a single layer of tissue at the location of abnormal organ function; wherein the single layer of tissue is a muscular layer of tissue; wherein the single layer of tissue is a mucosal layer of tissue; wherein the organ is a bladder, and wherein the treatment units are positioned on an outer surface of the bladder wall.

Yet another embodiment of the invention is directed to a method for treating a urinary system. The method may include positioning a first medical device with respect to an organ of the urinary system. The first medical device may include one or more first treatment units being configured to treat a tissue layer in a wall of the organ. The method may further include positioning a second medical device with respect to the organ. The second medical device may include one or more second treatment units. The method may also include treating the tissue layer with one of the first treatment units and the second treatment units.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates a system for detecting and treating a urinary tract condition including a medical device having a plurality of end effector units, according to an embodiment of the invention;

FIG. 2 is a cross-section of medical device along line 2-2 of FIG. 1;

FIG. 3A is a side view of an end effector assembly of the medical device of FIG. 1, according to an embodiment of the invention;

FIG. 3B is an exploded view of a portion of a leg of end effector assembly of FIG. 3A;

FIG. 3C is an end view of an individual end effector unit disposed on a leg of the distal assembly shown in FIG. 3B, according to an embodiment of the invention;

FIG. 4A illustrates a medical device being inserted into a bladder of a lower urinary tract of a body, according to an embodiment of the invention;

FIG. 4B illustrates a distal end of the medical device of FIG. 1 contacting a bladder wall to detect and/or treat abnormal bladder function, according to an embodiment of the invention;

FIG. 5A-E illustrate alternative configurations of the end effector assembly of the medical device of FIG. 1, according to embodiments of the invention;

FIG. 6 illustrates a medical device positioned on an outer bladder wall, according to a second embodiment of the invention;

FIG. 7A illustrates an alternative embodiment of the medical device of FIG. 6 positioned on an empty bladder, the medical device includes a first set of treatment elements spaced from a second set of treatment elements;

FIG. 7B illustrates an alternative embodiment of the medical device of FIG. 7A positioned on an expanded bladder; and

FIG. 8 illustrates another alternative embodiment of the medical device of FIG. 6.

### DESCRIPTION OF THE EMBODIMENTS

Reference will now be made in detail to exemplary embodiments of the invention, examples of which are illustrated in the accompanying drawings. Whenever possible, the same reference numbers will be used throughout the drawings to refer to same or like parts.

Embodiments of the invention relate generally to systems and methods for detecting and/or treating lower urinary tract conditions. More particularly, embodiments of the invention relate to systems and methods for detecting and/or treating bladder overactivity. Bladder overactivity is characterized by involuntary contractions of the detrusor muscle during bladder filling, which result in a sudden urge to urinate. The systems and methods described herein may be used to treat conditions of the body other than bladder overactivity such as, for example, bladder sphincter dyssynergia, stress incontinence, painful bladder syndrome (interstitial cystitis), nerve pain, hypertension, or arrhythmia.

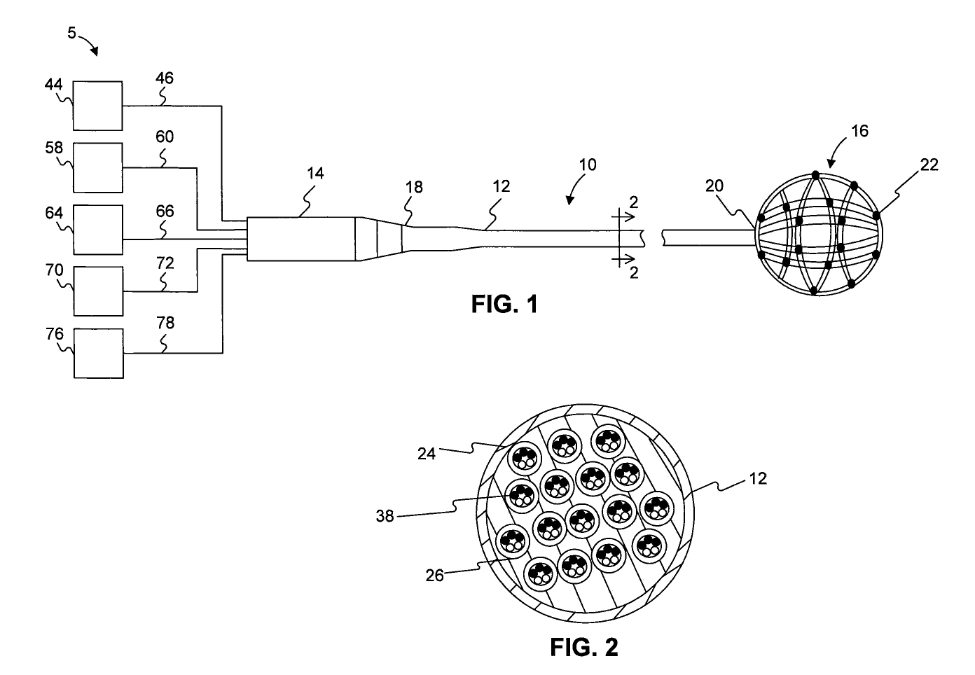

FIG. 1 illustrates an exemplary system 5 including a medical device 10. In one embodiment, system 5 may include a fluid source 44 and one of a signal processing device 58, an electrical energy source 64, a coolant source 70, and a laser source 76. In another embodiment, system 5 may include fluid source 44 and two of: signal processing device 58, electrical energy source 64, a coolant source 70, and laser source 76. For example, system 5 may include fluid source 44, coolant source 70 and electrical energy source 64; or source 44, coolant source 70 and laser source 76. In yet another embodiment, system 5 may include fluid source 44, coolant source 70 and at least two of: signal processing device 58, electrical energy source 64, and laser source 76. Fluid source 44, signal processing device 58, electrical energy source 64, coolant source 70, and laser source 76 are connected to medical device 10 by way of one or more fluid conduits 46, leads 60, wires 66, catheters 72, and optical fibers 78, respectively. It is contemplated that additional cooling lines may be provided to provide temperature control.

Medical device 10 may include an elongate member 12, a handle portion 14, and an end effector assembly 16. Elongate member 12 may have a proximal end 18 and a distal end 20. For purposes of this disclosure, “proximal” refers to the end closer to the device operator during use, and “distal” refers to the end further from the device operator during use. Handle portion 14 may be disposed at proximal end 18 of elongate member 12 and end effector assembly 16 may be disposed at distal end 20 of elongate member 12. End effector assembly 16 may include one or more end effector units 22 uniformly distributed over end effector assembly 16 to detect abnormal bladder function and deliver therapeutic treatment to the bladder.

FIG. 2 is a cross-section of elongate member 12 along lines 2-2 in FIG. 1. Elongate member 12 may be a solid rod or tube, made from any suitable biocompatible material known to one of ordinary skilled in the art having sufficient flexibility to traverse a urinary tract. Such materials may include, but are not limited to, rubber, silicon, synthetic plastics, stainless steel, metal-polymer composites, and metal alloys of nickel, titanium, copper cobalt, vanadium, chromium, and iron. In one embodiment, the material forming elongate member 12 may be a superelastic material such as nitinol, which is a nickel-titanium alloy. Elongate member 12 may have any cross-sectional shape and/or configuration and may be any desired dimension that can be received in the lower urinary tract. An outer sheath 24 may surround elongate member 12. Outer sheath may be constructed from an insulating polymer material such as polymide, polyurethane, or any other suitable material.

Elongate member 12 may include one or more lumens 26 extending from proximal end 18 of the elongate member 12 to distal end 20 of the elongate member 12. It is to be understood that lumens 26 may have any size, cross-sectional area, shape, and/or configuration. Although the depicted embodiment includes sixteen lumens, elongate member 12 may include a greater or lesser number of lumens 26. It is to be understood that the number of lumens 26 may depend on the number of end effector units 22 on end effector assembly 16.

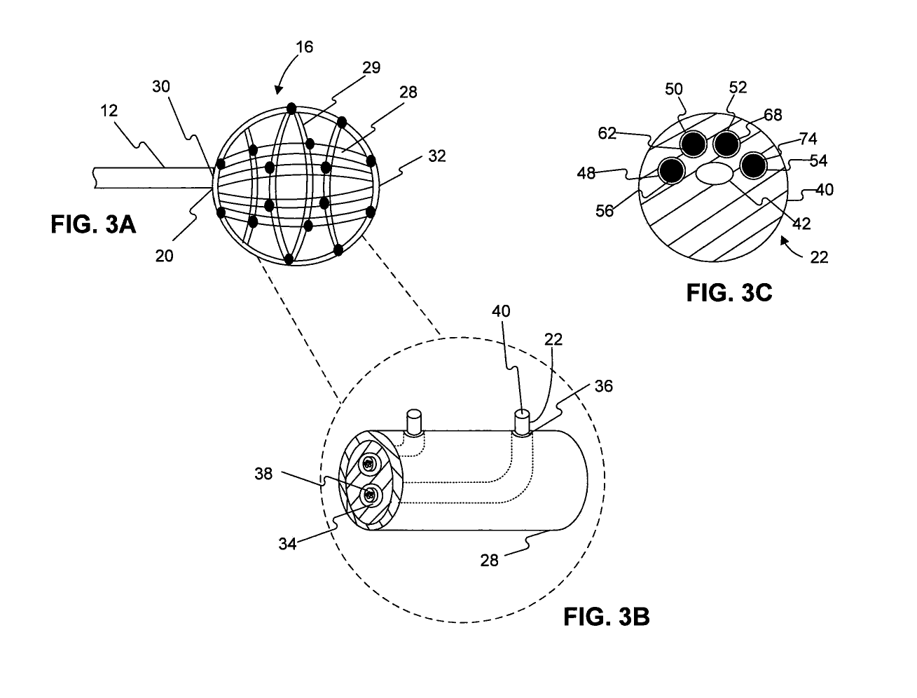

FIG. 3A depicts a side view of end effector assembly 16. As shown in FIG. 3A, end effector assembly 16 may extend distally from distal end 20 of elongate member 12, and may include a plurality of legs 28 extending from a proximal end 30 of end effector assembly 16 to a distal end 32 of end effector assembly 16. In some embodiments, end effector assembly 16 may also include one or more circumferentially extending legs, such as legs 29. In this disclosure, descriptions of legs 28 also pertain to legs 29, and vice versa.

End effector assembly 16 may be made out of the same piece of material as elongate member 12. Alternatively, end effector assembly 16 may be fabricated independently by any known means and may be made integral with elongate member 12 through connection of a proximal end 30 of the end effector assembly 16 to a region of elongate member 12, such as the distal end 20 of elongate member 12. Connection of proximal end 30 of end effector assembly 16 may be accomplished through any suitable means of fixedly connecting end effector assembly 16 to elongate member 12. For example, possible connections may include, but are not limited to welding, soldering, and/or crimping.

End effector assembly 16 may have any shape and/or configuration and may be any desired dimension that can be received in the lower urinary tract. In the exemplary embodiment shown in FIG. 3A, legs 28 are configured so that end effector assembly 16 forms a three-dimensional sphere in an expanded state. Legs 28 may be constructed from a material such as, for example, a shape memory metal alloy or a polymer so that legs 28 may collapse to have a smaller cross-section in a collapsed state. It will be understood that end effector assembly 16 may form any other shape and or configuration in the expanded state and the collapsed state.

Although FIG. 3A, shows that that end effector assembly 16 comprises six legs 28 extending from proximal end 30 of end effector assembly 16 to a distal end 32 of end effector assembly 16 (and four circumferential legs 29), end effector assembly 16 may include any number of legs 28 (or 29) having any desired pattern and/or configuration. For example, legs 28 may be cylindrical, square, semi-circular, rectangular, or any other suitable shape. In addition, legs 28 may be any cross-sectional shape known in the art including, but not limited to, circular, square, or ovular.

Each leg 28 of end effector assembly 16 may include one or more lumens 34 located longitudinally therein. Lumens 34 may have any size, cross-sectional area, shape, and/or configuration. Each lumen 34 may be in communication with a corresponding lumen 26 of elongate member 12, and may extend from proximal end 30 of end effector assembly 20 to an exit aperture 36 on leg 28.

Within each lumen 26 of elongate member 12 and the corresponding lumen 34 of the end effector assembly 16 is a bundle of wires, optic fibers, and/or fluid conduits 38 that terminate at an individual end effector unit 22. More particularly, each bundle 38 may include one or more of a fluid conduit 46 associated with fluid source 44, at least one wire 60 associated with signal processing device 58, at least lead 66 associated with electrical energy source 64, at least one catheter 72 associated with coolant source 70, and at least one optical fiber 78 associated with laser source 76, that terminate at an individual end effector unit 22. In some embodiments, bundle 38 may be a flexible or polymer circuit having sufficient flexibility to traverse lumen 26 of elongate member and lumen 34 of end effector assembly 16.

Each end effector unit 22 may be fixed in exit aperture 36 and extend outwardly from an exterior surface of leg 28. A surface 40 of each end effector unit 22 may be configured to contact tissue. In some embodiments, end-effector units 22 may define a conical shape having a flat surface 40. In other embodiments, end effector units 22 may be tapered. Other shapes, sizes and/or configurations of end effector unit 22 and/or surface 40 are also contemplated. It is contemplated that, in some embodiments, each end effector unit 22 may move relative to exit aperture 36. In those embodiments, each end effector unit 22 may be a needle configured to penetrate tissue.

FIG. 3C is an end view of an individual end effector unit 22. As shown in FIG. 3C, each end effector unit 22 may include a fluid port 42. Fluid port 42 may have any size, shape, and/or configuration. Fluid port 42 may be configured to deliver therapeutic fluids from the fluid source 44 to tissue adjacent end effector unit 22. More particularly, fluid conduit 46 may extend from fluid port 42 of end effector unit 22 proximally through lumen 34 of leg 28 and a corresponding lumen 26 of elongate member 12 in bundle 38 to fluid source 44. With this arrangement, therapeutic, diagnostic, or other fluids may be circulated between fluid source 44 and fluid port 42 of end effector unit 22. Therapeutic fluids may include, for example, growth factors that promote tissue healing such as, for example, keratinocyte growth factor. Alternatively, the fluids may be an anti-infective agents or anesthetic.

Each end effector unit 22 may further include one or more apertures, separate from fluid port 42. In one embodiment, end effector unit 22 may include a first aperture 48, a second aperture 50, a third aperture 52, and a fourth aperture 54. Although the depicted embodiment of end effector unit 22 includes four apertures, end effector unit 22 may include a greater or lesser number of apertures. Apertures may have any size, shape, and/or configuration. For example, in the exemplary embodiment shown in FIG. 3C, each of first aperture 48, second aperture 50, third aperture 52, and fourth aperture 54 has a substantially circular cross-section.

A sensing element 56 may be provided in first aperture 48. Sensing element 56 may be flush with, or protrude from, surface 40 of end effector unit 22. Sensing element 56 may encompass physical, mechanical, chemical, electrical, and biochemical sensors, and may be of a type and kind well known in the art. Although the depicted embodiment includes a single sensing element 56, it is contemplated that a greater or lesser number of sensing elements 56 may be provided. It is further contemplated that in some embodiments, sensing element may be an imaging unit that, for example, includes a source for emitting light at a wavelength sufficient to induce fluorescence of tissue and sensors that are capable of detecting light at a wavelength at which tissue fluoresces.

Sensing element 56 may be configured to detect one or more indicators of abnormal bladder function such as, for example, abnormal detrusor contractions or excitability of a distinct anatomical area of the bladder. Sensing element 56 may transmit the measured information proximally to a signal processing device 58. More particularly, a wire 60 may extend from sensing element 56 proximally through lumen 34 of leg 28 and a corresponding lumen 26 of elongate member 12 in bundle 38 to signal processing device 56. Signal processing device 58 may be configured to process the information using methods and procedures known to one of ordinary skill in the art. Signal processing device 58 may be further configured to identify the location of abnormal bladder function and, more particularly, the site where the abnormal bladder function originates.

Treatment elements may be provided in each of second aperture 50, third aperture 52, and fourth aperture 54. Treatment elements may either be fixed to or more relative to second aperture 50, third aperture 52, and fourth aperture 54. Treatment elements may be any suitable energy transmission medium known to one of ordinary skill in the art which operates by delivering energy such as, for example, thermal energy, microwave energy, radiofrequency energy, or laser energy, to a selected anatomical site to cause tissue necrosis. Such devices may include, but are not limited to, radio frequency (RF) devices, cryoablation catheters, lasers, microwave probes, thermoelectric cooling devices, and ultrasonic ablation devices and other devices capable of heating or cooling tissue. In some embodiments, treatment elements may have a needle body and a device for heating or cooling tissue disposed at the distal end of the needle body. In the preferred embodiment, an RF electrode 62 may be provided in second lumen 50, a cryoablation tip 68 may be provided in third aperture 52, and a distal end 74 of optical fiber 78 may be provided in fourth aperture 54. It is to be understood, however, that end effector unit 22 may include a greater or lesser number of treatment elements.

RF electrode 62 may be provided in second lumen 50 so that RF electrode 62 is flush with, or protrudes from, distal facing surface 40 of end effector unit 22. RF electrode 62 may be connected to an electrical energy source 64 (FIG. 1) such as, for example, an RF generator, to deliver electrical energy to tissue adjacent to end effector 22. More particularly, a wire 66 may extend from RF electrode 62 proximally through lumen 34 of leg 28 and corresponding lumen 26 of elongate member 12 in bundle 38 to electrical energy source 64. With this arrangement, RF energy can be transmitted from electrical energy source 64 to RF electrode 62.

Cryoablation tip 68 may be provided in third lumen 52 of end effector unit 22 such that cryoablation tip 68 is adjacent to, flush with, or protrudes from, distal facing surface 40 of end effector unit 22. Cryoablation tip 68 may be connected to a coolant source 70 (FIG. 1) so as to direct a flow of a coolant to distal surface 40 and remove heat from tissue adjacent to end effector 22. More particularly, a catheter 72 may extend from cryoablation tip 68 proximally through lumen 34 of leg 28 and corresponding lumen 26 of elongate member 12 in bundle 38 to coolant source 70. With this arrangement, coolant fluid may be circulated between coolant source 70 and cryoablation tip 68.

A distal end 74 of an optic fiber 78 may be provided in third lumen 54 of end effector unit 22 such that distal end 74 of optic fiber 78 is adjacent to, flush with, or protruding from distal facing surface 40 of end effector unit 22. Optic fiber 74 may be connected to a laser source 76 (FIG. 1) to deliver laser energy to tissue adjacent end effector 22. More particularly, optic fiber 74 may extend from end effector 22 proximally through lumen 34 of leg 28 and corresponding lumen 26 of elongate member 12 in bundle 38 to laser source 76. With this arrangement, laser energy can be transmitted from laser source 76 to distal end 74 of optic fiber 78.

In some additional embodiments, an imaging device (not shown) may be provided on each end effector unit 22. The imaging device may be a camera, lens, digital imaging chip (e.g., a CCD or CMOS chip), or other image receiving device. The imaging device may be connected to a control device (not shown) which may transmit signals using a fiber optic or another type of cable.

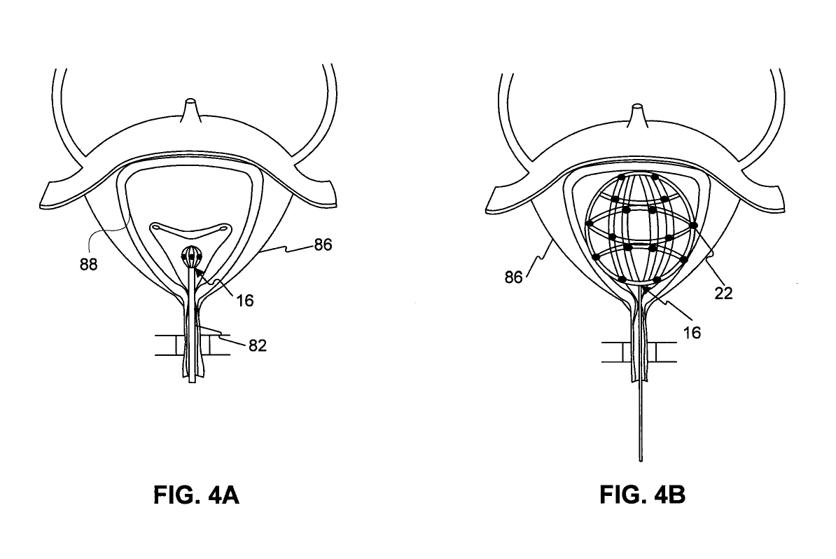

FIGS. 4A and 4B illustrate a method for detecting and/or treating a urinary tract condition such as, for example, bladder overactivity. Referring to FIG. 4A, medical device 10 may be inserted into the urethra of a patient for access to the internal sphincter, trigone, neck, or dome of a bladder 86. In some embodiments, when medical device 10 may be placed against an outer bladder wall, surgical access for placement against an outer bladder wall may be achieved using pelvic floor repair procedure or laparoscopic techniques. It is to be understood that, in addition to the bladder of the lower urinary tract, medical device 10 may be used in any visceral organ to detect and/or treat abnormal organ function.

It is contemplated that medical device 10 may be used for diagnostic and treatment purposes during a procedure. Alternatively, medical device 10 may be implanted temporarily or permanently within the bladder and end effector assembly 16 may communicate with a remote data processing unit wirelessly. For example, end effector assembly 16 may contain wireless sensing units configured to detect one or more indicators of abnormal bladder function and wireless treatment units configured to delivery energy to the bladder (e.g., wireless RF electrode).

In one embodiment, medical device 10 may be advanced to bladder 86 through an access sheath 82. Once a distal end of access sheath is positioned in bladder 86, distal assembly 16 may be advanced distally out of sheath 82 so that distal assembly 16 may expand. For example, this may be achieved by pulling sheath 82 proximally relative to elongate member 12. Any suitable handle portion 18 may be used to effect deployment and expansion of end effector assembly 16. In an alternative embodiment, a balloon (not shown) may be placed in bladder 86 to distend bladder 86 so that end effector assembly 16 may expand. When fully expanded, end effector assembly 16 may have a substantially spherical shape such that surface 40 of each end effector unit 22 contacts an interior bladder wall 88 (FIG. 4B).

In some embodiments, device operator may uniformly deliver energy to bladder 86 after positioning surfaces 40 of end effector units 22 adjacent to interior bladder wall 88. Energy may be delivered through the same treatment elements (i.e., RF electrode 62, cryoablation tip 68, distal end 74 of optic fiber 78, or any other temperature-controlled heating or cooling element) on each end effector unit 22 to uniformly treat tissue of bladder 86.

In other embodiments, sensing element 56 may be used to detect one or more indicators of abnormal bladder function. In one embodiment, each sensing element 56 may be configured to detect abnormal detrusor contractions. Additionally and/or alternatively, each sensing element 56 may be configured to detect the excitability of a distinct anatomical area of the bladder.

Each sensing element 56 may transmit the measured information to a signal processing device 58. Signal processing device 58 may then determine the origin of abnormal bladder function by methods and procedures known to one of ordinary skill in the art. In one embodiment, signal processing device 58 may map electrical activity of the bladder to determine the origin of abnormal bladder function.

After determining the location of the origin of abnormal bladder function, one or more treatment elements disposed on the end effector unit 22 located at the site at which abnormal bladder function originates may be activated to deliver energy to tissue adjacent the end effector unit 22. In some embodiments, the device operator may selectively deliver electrical energy through RF electrode 62 to tissue adjacent to end effector unit 22. The device operator may alternatively and/or additionally remove heat from tissue adjacent to end effector 22 through cryoablation tip 68. The device operator may alternatively and/or additionally deliver laser energy through distal end 74 of optic fiber 78 to tissue adjacent end effector unit 22.

Energy may be delivered at varying durations to achieve a range of effects from disrupting spontaneous detrusor contractions to inducing tissue shrinkage, collagen/elastin denaturation, or cellular necrosis. Additionally and/or alternatively, energy may be delivered at varying temperatures to effect cellular necrosis. For example, energy may be delivered at 55° C. and above to cause cell necrosis or at 45° C. to cause modification without necrosis. In other embodiments, energy may be delivered via cryoablation tip 68 at about 37° C. for desired cell modification. The frequency, duration, and/or temperature of energy delivered to tissue adjacent end effector unit 22 may be determined based on the desired cell modification.

The therapy, including the form of energy delivered, frequency, duration, depth of ablation, and adjustment of temperature, may also be determined by the device operator based on the type of tissue at the site at which the abnormalities originate. For example, therapy may differ between treatment of skeletal muscle and treatment of smooth muscle cells. Additionally, therapy may be different for the treatment of the urothelium layer. The therapy may also differ to treat different thicknesses of the bladder wall.

Therapeutic fluids may be delivered to the tissue adjacent end effector unit 22 after treatment. Therapeutic fluids may be delivered via fluid conduit 42 which may facilitate urothelial healing.

In some additional embodiments, each end effector unit 22 may be configured to selectively treat a targeted tissue layer of the bladder wall at the location of abnormal function. In particular, it may be beneficial to selectively treat only the mucosal layer of the bladder wall at the location of abnormal function avoiding treatment or damage to one or more of the submucosa, muscularis, and serosa. In these embodiments, the device operator may selectively control the depth and/or the temperature of energy delivered from the one or more treatment element of end effector unit 22 to target the mucosal tissue layer in the bladder wall.

For example, in one embodiment, the device operator may control the depth of thermal treatment by delivering laser energy through distal end 74 of optic fiber 78 to tissue adjacent an end effector unit 22. The laser energy may be at a wavelength sufficient to target only the mucosal layer in the bladder wall. In another embodiment, the device operator may deliver electrical energy through one or more RF electrodes 62 to tissue adjacent to end effector unit 22. In one embodiment, the one or more RF electrodes may include a bipolar RF electrode array (see e.g., Halo Technology™ developed by Barrx Medical). In yet another embodiment, the device operator may deliver a fluid such as, for example, water or saline, through fluid port 42 at a predetermined temperature so as to achieve thermal treatment at a desired depth of tissue, including for example only the mucosal layer. It is contemplated that the fluid may be delivered through one or more tubes positioned in a separate aperture on end effector unit 22.

Additionally and/or alternatively, the energy may be delivered at a low temperature in order to selectively treat only the mucosal layer. For example, energy may be delivered at 42° C. and below to prevent tissue and nerve damage. In some embodiments, energy may be delivered at approximately 41°±0.5° C. In these embodiments, the sensing element 56 may include a thermal sensor to measure the temperature of the mucosal layer and/or the temperature of the energy delivered. It is contemplated that the thermal sensor may be separately provided. In such an embodiment, the thermal sensor may be provided on a needle that may be inserted into the bladder wall and positioned at the intersection between the mucosal layer and the muscular layer so as to sense the temperature of energy delivered to the mucosal layer.

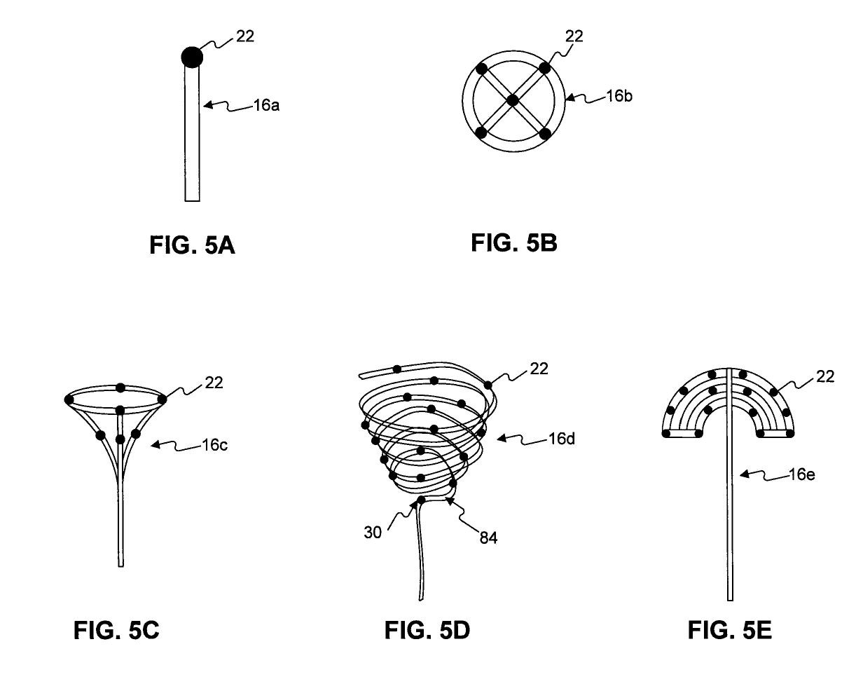

Alternative non-limiting examples of end effector assemblies having various shapes and/or distal configurations are shown in FIGS. 5A-5E.

FIGS. 5A and 5D depict end effector assemblies having wire configurations. In particular, end effector assembly 16b, as shown in FIG. 5A, may have a substantially linear configuration. A single end effector unit 22 may be disposed at distal end 32 of end effector assembly 16a. In another embodiment, end effector assembly 16d, as shown in FIG. 5D, may have a helical configuration preferably tapering from a larger diameter at a distalmost end thereof to a smaller diameter proximally of the distalmost end thereof. A kink 84 may be disposed adjacent proximal end 30 of end effector assembly 16d.

FIG. 5C depicts a medical device including end effector assembly 16c having a plurality of legs curving away from a longitudinal axis of end effector assembly 16c.

FIGS. 5B and 5E depict end effector assemblies having a mesh configuration. In particular, end effector assembly 16b, as shown in FIG. 5B, may have a circular shape. And in yet another embodiment, end effector assembly 16e, as shown in FIG. 5E, may have a semi-circular shape. End effector assemblies 16c and 16e may be additionally planar, concave, or convex.

Distal end configurations shown in FIGS. 1 and 5A-5E may facilitate placement of end effector units 22 at a selected anatomical area of the bladder or other organ. For example, in one embodiment, end effector assembly 16 as shown in FIG. 1 may facilitate placement adjacent an interior bladder wall 88. In another embodiment, end effector assemblies 16a and 16d, shown in FIGS. 5A and 5D, may facilitate placement in the internal sphincter. In yet another embodiment, end effector assemblies 16b and 16e, shown in FIGS. 5B and 5E, may facilitate placement adjacent the outer bladder wall. And in yet another embodiment, end effector assembly 16c may facilitate placement adjacent the trigone.

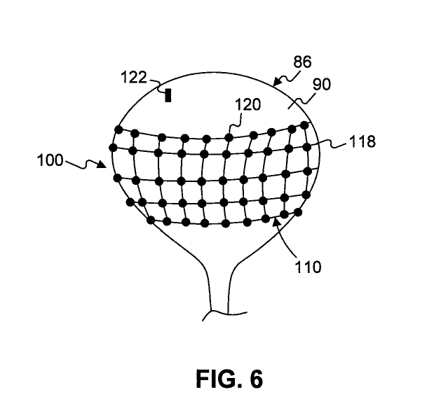

FIG. 6 depicts an exemplary medical device 100 and the components thereof in accordance with a second embodiment of the invention. As illustrated in FIG. 6, medical device 100 may include a support structure 110 and a plurality of treatment elements 120 uniformly disposed on support structure 110 to deliver therapeutic treatment to a targeted tissue layer of bladder wall 90. In some embodiments, medical device 100 may be configured to deliver energy to only a muscular layer of the bladder wall 90. In other embodiments, medical device 100 may be configured to delivery energy to only a mucosal layer of bladder wall 90. In yet other embodiments, medical device 100 may be configured to deliver energy to both a muscular layer and a mucosal layer in order to decrease spontaneous bladder contractions.

Support structure 110 may have any size, shape, and/or configuration capable of generally conforming to bladder 86. In the exemplary embodiment, support structure 110 may be a one-piece structure configured to extend completely around an outer surface of bladder wall 90 to surround a portion of bladder 86. It is contemplated, however, that support structure 110 may have any other size and/or configuration to conform to any other portion of bladder 86 including the interior surface of bladder wall 90. It is further contemplated that support structure 110 may not extend around the entire circumference of bladder 86, and may only extend around a portion of the circumference of bladder 86.

In one embodiment, support structure 110 may include a plurality of filaments 118 arranged in an open mesh configuration. Filaments 118 may be constructed from any suitable biocompatible material having elastic and recoil properties including, but not limited to, rubber, silk, synthetic plastics, stainless steel, metal-polymer composites, or metal alloys. In some embodiments, filaments 118 may be formed of conductive polymers or thermal conductive polymers configured to generate and apply heat to tissue of bladder 86 when supplied with electrical current. It is further contemplated that filaments 118 may be cast from conductive and non-conductive polymers so that selective portions of filaments 118 generate heat.

Filaments 118 may be constructed to have sufficient flexibility and strength to maintain the position of support structure 110 on the outer surface of bladder wall 90. Portions of filaments 118 may additionally and/or alternatively include adhesive materials so as to adhere support structure 110 to bladder wall 90. Other retaining structures are also contemplated.

An array of treatment elements 120 may be attached to or embedded in support structure 110 so that treatment elements 120 are position adjacent bladder 86. In the exemplary embodiment illustrated in FIG. 6, treatment elements 120 are disposed at the intersection of filaments 118. While the depicted embodiment includes 50 treatment elements, it is contemplated that a greater or lesser number of treatment elements may be provided. Treatment elements 120 may be any suitable energy transmission medium known to one of ordinary skill in the art which operates by delivering energy such as, for example, thermal energy, microwave energy, radiofrequency energy, or laser energy, to treat tissue of bladder 86. Such devices may include, but are not limited to, radio frequency (RF) devices, lasers, microwave probes, ablation devices, and other devices capable of heating tissue.

In a preferred embodiment, treatment elements 120 may be electrodes. Electrodes may be connected to a source of electrical energy (not shown) to deliver thermal energy to bladder 86. In one embodiment, filaments 118 and a wire (not shown), may provide an electrical pathway from an energy source such as, for example, an implantable generator, to each end effector unit 120. The generator may be implanted adjacent the scrotum, the buttocks, or within the abdominal musculature. In another exemplary embodiment, treatment elements 120 may wirelessly communicate with a source of electrical energy. In some embodiments, treatment elements 120 may be switchable electrodes to provide controlled delivery of thermal energy to bladder 86. In these embodiments, electrodes may be activated based on a sensed condition of the bladder such as, for example, bladder filling.

In other embodiments, treatment elements 120 may be optical elements. Optical elements may be connected to a source of energy to apply heat to tissue of bladder 86. In one embodiment, filaments 118 may be optical fibers connected to a source of laser energy. In this embodiment, the treatment element may be the distal end of the optical fibers. In another embodiment, the optical elements may be LEDs directly or wirelessly connected to a source of electrical energy. In these embodiments, each optical element may be configured to deliver energy to the tissue of bladder 86 at a specific wavelength. Additionally and/or alternatively, the energy emitted from treatment elements 120 may be sufficient to activate a photothermal dye injected into the tissue of bladder 86. The photothermal dye may be, for example, chromophore palladium(II) octabutoxynaphthalocyanine (PdNc(OBu)8), or any other known photothermal dye.

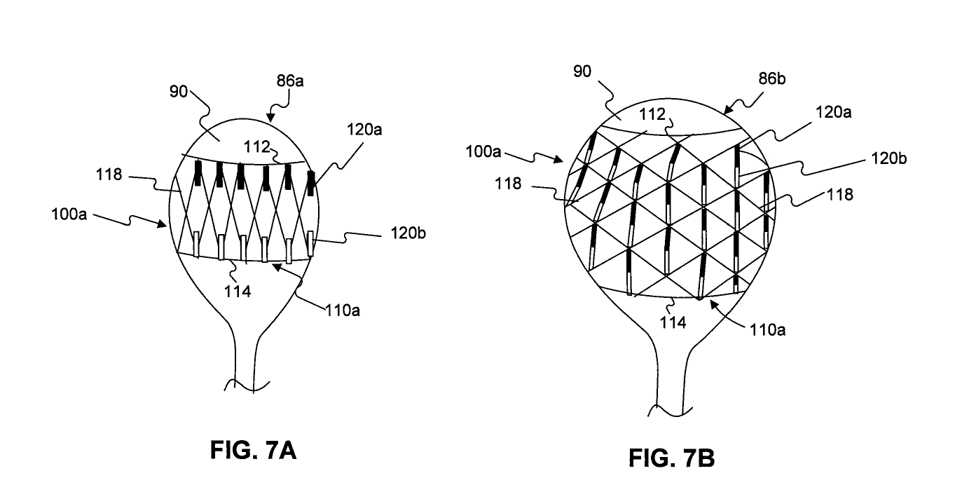

FIG. 7A illustrates an alternative embodiment of the medical device shown in FIG. 6. In this embodiment, medical device 100a includes a support structure 110a having one or more filaments 118a extending between a first edge 112 and a second edge 114 of support structure 110a. Medical device 100 may also include a first set of treatment elements 120a and a second set of treatment elements 120b attached to or embedded in support structure 110a between first edge 112 and second edge 114. First set of treatment elements 120a and second set of treatment elements 120b may be conductive elements configured to deliver energy when first set of treatment elements 120a contact second set of treatment elements 120b. In some embodiments, first set of treatment elements and second set of treatment elements may be monopolar or bipolar electrodes. While the depicted embodiment illustrates a single row of the array, multiple rows are contemplated, as shown in FIG. 7B.

First set of treatment elements 120a and second set of treatment elements 120b may be positioned on filaments 118a so that first set of treatment elements 120a are spaced from second set of treatment elements 120b when bladder 86 is empty, as shown in FIG. 7A. As bladder 86 expands, filaments 118 may pivot relative to first edge 112 and second edge 114 to bring first edge 112 closer to second edge 114. In this manner, first set of treatment elements 120a are brought into contact with the second set of treatment elements 120b, as shown in FIG. 7B. Energy may be delivered through both the first set of treatment elements 120a and second set of treatment elements 120b when first set of treatment elements 120a contact second set of treatment elements 12b to treat tissue of bladder 86.

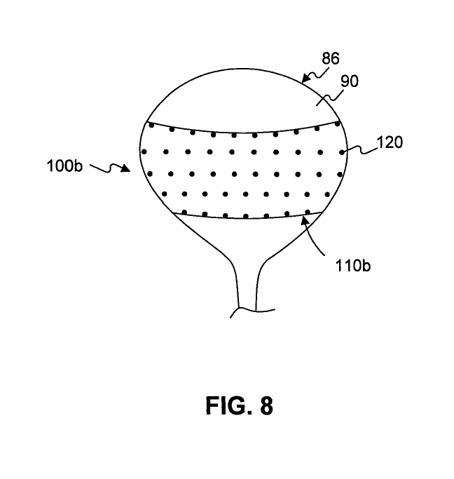

Another alternative embodiment of the medical device of FIG. 6 is shown in FIG. 8. In this embodiment, medical device 100b may include a support structure 110b. Support structure 110b may be, for example, a continuous or porous polymer sheet having an array of treatment elements 120 embedded therein. Support structure 110b may be constructed from flexible and/or elastic materials including, but are not limited to, elastomers, silicone, polytetrafluoroethylene (PTFE), polyethylene terephthalate (PET), fluorinated ethylene propylene (FEP), polypropylene, polyurethanes and their copolymers, or thin films such as latex. In some embodiments, it is contemplated that sheet may be constructed from conductive polymers and/or thermally conductive polymers that generate heat. Such polymers include, but are not limited to, ABS, nylon, liquid-crystal polymers (LCP), and polyetheretherketone (PEEK). In some embodiments, treatment elements 120 may be nanoparticle-sized electrodes embedded in support structure 110b. In other embodiments, support structure 110b may be a flexible circuit having treatment elements 120 formed therein. In these embodiments, treatment elements 120 may either be connected to a source of energy (e.g., implanted generator) or may be wirelessly in communication with a source of energy.

Referring to back to FIG. 6, a method of treating a urinary tract condition will now be described. Medical device 100 may be inserted in the body and positioned on bladder 86 so that support structure 110 may conform to bladder 86 and treatment elements 120 may be positioned adjacent bladder wall 90. The position of support structure 110 may be determined based on the anatomical site and/or tissue layer targeted for treatment. For example, it may be desired to selectively treat only the muscular layer of bladder wall 90 (i.e., the detrusor muscle). In those embodiments, support structure 110 may be positioned on an outer surface of bladder wall 90 extending around a middle portion of bladder 86 where the detrusor muscle is relatively thick and nerves are relatively less perfuse. It is contemplated, however, that support structure 110 may be positioned at other locations on the outer surface of bladder wall 90 to treat tissue layer or nerves in, for example, the trigone. It is further contemplated that support structure110 may be positioned on an interior surface of bladder wall 90 to treat, for example, the mucosal layer of bladder wall 90. Surgical access for placement against the exterior bladder wall 90 may be achieved using pelvic floor repair procedure or laparoscopic techniques. Surgical access for placement against the interior bladder wall 90 may be achieved using endourologic or laparoscopic techniques.

In some embodiments, medical device 100 may be implanted temporarily to provide thermal treatment during the course of a procedure. In other embodiments, medical device 100 may be implanted permanently to provide continuous treatment to tissue of bladder 86. In those embodiments, medical device may communicate with an implanted generator or a remote data processing unit wirelessly to delivery energy to bladder 86.

After positioning medical device 100 on bladder 86, treatment elements 120 of medical device 100 may contact bladder wall 90 and deliver energy to bladder wall 90. In some embodiments, energy may be delivered simultaneously through the entire array of treatment elements 120 to uniformly treat bladder wall 90. In other embodiments, energy may be delivered through one or more selected treatment elements 120 to treat bladder wall 90 at specific anatomical locations.

In some embodiments, energy may only be delivered when bladder 86 is expanding so as to control the frequency of spontaneous bladder contractions. In these embodiments, a sensing element 22 may be provided to sense a condition such as, for example, the volume of fluid within bladder 86 or the pressure within bladder 86. Sensing element 22 may be a physical, chemical, electrical, or biochemical sensor, and may be of a type and kind well known in the art. Treatment elements 120 may be activated to deliver energy to tissue adjacent treatment elements 120 when the bladder 86 starts to fill, and may not deliver energy when bladder 86 is empty. In some embodiments, treatment elements 120 may be activated to deliver energy when bladder 86 reaches approximately 200-300 mL. After treatment elements 120 have been activated, treatment elements 120 may deliver energy continuously or periodically until bladder 86 has been emptied.

Energy may be delivered to heat targeted tissue layers to achieve a range of effects from disrupting spontaneous contractions to inducing tissue shrinkage, collagen/elastin denaturation, or cellular necrosis. For example, applying energy to heat only the muscular layer of bladder wall 90 (i.e., detrusor muscle), only the mucosal layer of bladder wall 90, or a combination of both the muscular layer and the mucosal layer may have certain benefits including, for example, disrupting spontaneous bladder contractions. In some embodiments, delivering thermal energy so as to increase the local temperature in the muscular layer to a temperature between 37° C. and 43° C. may, for example, disrupt spontaneous bladder contractions. Additionally and/or alternatively, delivering thermal energy so as to increase the local temperature of the mucosal layer to a temperature between 37° C. and 43° C. and, more preferably, to a temperature of 43° C., may disrupt spontaneous bladder contractions. At these temperatures, other tissue damage and nerve damage may also be avoided. In other embodiments, delivering thermal energy so as to increase the local temperature in the trigone to a temperature of about 41° C. may damage the nerves so as to disrupt spontaneous bladder contractions. In embodiments where energy is delivered periodically, the frequency and/or duration of the thermal treatment may be determined based the temperature of the treated tissue layer. For example, energy may be delivered at longer durations and/or higher frequency at lower temperatures and shorter durations and/or lower frequency at higher temperatures. It is contemplated that that the local temperature may be measured by a temperature sensor embedded in or separate from medical device 100.

In some embodiments, it may be desirable to increase the local temperature of both the mucosal and muscular tissue layers between 37° C. and 43° C. In those embodiments, it is contemplated that a first medical device 100 may be positioned adjacent outer bladder wall 90 and a second medical device 100 may be positioned adjacent an interior bladder wall. The first medical device may be positioned on the outer surface of bladder wall 90 to apply heat to the muscular layer, and the second medical device may be positioned on the interior surface of bladder wall 90 to apply heat to the mucosal layer. The second medical device may be secured against the interior surface of bladder wall by, for example, sutures or other retaining devices.

In other embodiments, medical device 100 may be positioned adjacent an outer bladder wall 90 and medical device 10 may be positioned adjacent an interior bladder wall 90, or vice versa, to treat tissue located at various anatomical sites of bladder 86 and/or target various tissue layers.

In yet other embodiments, one or more treatment elements may be embedded within the tissue of, bladder wall 90 between the mucosal layer and the muscular layer in bladder wall 90 by injection or dissection to simultaneously heat both targeted tissue layers. In this embodiment, the treatment elements may be electrodes and/or conductive polymers or thermally conductive polymer devices. It is contemplated that treatment elements may be uniformly dispersed through bladder wall 90 or may only be positioned at distinct anatomical sites such as, for example, the origin of abnormal function.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A method for treating a urinary system, comprising:
inserting a medical device within an organ of the urinary system, the medical device comprising:
an elongate member having a proximal end and a distal end; and
an end effector assembly extending distally from the distal end of the elongate member, the end effector assembly including a plurality of end effector units each having a sensing element for detecting a location of abnormal function and a treatment element for treating the location of abnormal function; and
positioning the medical device adjacent tissue of the organ of the urinary system; and
treating the location with the treatment element.

2. The method of claim 1, further including detecting a location of abnormal function.

3. The method of claim 1, further including contacting tissue with the plurality of end effector units.

4. The method of claim 1, wherein the step of treating the location includes delivering energy through all of the end effector units.

5. The method of claim 1, further comprising delivering a fluid to the location.

6. The method of claim 1, further including selectively treating only a single layer of tissue at the location of abnormal function.

7. The method of claim 6, wherein the layer of tissue is a mucosal layer of tissue.

8. The method of claim 1, further including delivering energy from the treatment element, and controlling a depth of thermal treatment to target only a single layer of tissue at the location of abnormal function.

9. The method of claim 1, further including delivering energy from the treatment element, and controlling a temperature of thermal treatment to target a layer of tissue at the location of abnormal function.

10. The method of claim 1, further including delivering energy from the treatment element at a predetermined temperature and targeting a desired depth of tissue.

11. A method for treating a urinary system, comprising:
inserting a medical device within the urinary system, the medical device comprising:
a support structure configured to conform to a portion of the organ; and
a plurality of treatment elements disposed on the support structure for treating a location of abnormal organ function; and
positioning the medical device with respect to an organ so that the plurality of treatment elements contact the organ; and
treating the location with one or more of the treatment elements.

12. The method of claim 11, further including delivery energy through all of the treatment elements.

13. The method of claim 11, further including delivering energy through the treatment elements to treat only a single layer of tissue at the location of abnormal organ function.

14. The method of claim 13, wherein the single layer is a muscular layer.

15. The method of claim 13, wherein the single layer is a mucosal layer.

16. The method of claim 11, further including delivering energy through the treatment elements to increase a local temperature of only a single layer of tissue at the location of abnormal organ function.

17. A method for treating a urinary system, comprising:
positioning one or more treatment units with respect to an organ wall, the one or more treatment units being configured to deliver thermal energy; and
delivering energy through the one or more treatment units to heat tissue of the organ wall.

18. The method of claim 17, further including heating only a single layer of tissue at the location of abnormal organ function

19. The method of claim 18, wherein the single layer of tissue is a muscular layer of tissue.

20. The method of claim 17, wherein the single layer of tissue is a mucosal layer of tissue.
